(19) United States
(12) Patent Application Publication
(10) Pub. No.: US 2009/0171439 A1
(43) Pub. Date: Jul. 2, 2009

(54) Temporary Stent

(76) Inventor: Thomas Nissl, Garstedt (DE)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/995,434

(22) PCT Filed: Jul. 12, 2006
(86) PCT No.: PCT/EP06/06815

§ 371 Date: Jul. 16, 2008

(30) Foreign Application Priority Data
Jul. 14, 2005 (DE) 10 2005 033 536.5
Apr. 13, 2006 (DE) 10 2006 017 873.4

(51) Int. Cl. A61F 2/06

(52) U.S. Cl. 623/1.15; 623/1.17

(57) ABSTRACT

Temporary stent having a plurality of ring elements arranged adjacent to each other, with said stent being capable of being explanted after it has been implanted in a body lumen, with all ring elements (3a, b, c, . . . ) starting out from a spine (2) extending over the length of the stent (1) and comprising at least two weakened areas (4, 5, 6) which enable the ring elements (3a, b, c, . . . ) to be collapsible during the process of explantation of the stent, and said spline (2) being provided with a recovery element (8) at its proximal end (7).

The invention relates to a temporary stent having a multitude of ring elements extending over the length of the stent which is capable of being explanted after having been implanted in a body lumen. The stent is particularly suited for vascular, that is coronary or peripheral blood vessels for example, but when appropriately sized may also be employed for other body lumens.

A multitude of intraluminal stent design types are known which are of reduced diameter to enable the stents to be transported to the placement site where they are to be implanted and expanded to the desired final diameter. Stents of this type are as a rule intended for permanent implantation, i.e. they shall remain in the body at the placement site for good.

Nevertheless, there are numerous case patterns where the implanted stent loses its function after a certain period of time—having fulfilled its purpose, for example widening a vessel, stabilizing a malfunction or obstructing a fistula or branch, so that it is no longer needed.

There are other cases where it becomes necessary to remove a placed stent which, for example, has been wrongly placed or due to incompatibility reactions. More often than not, removing a placed stent is a complex undertaking and a not at all riskless process. Especially if coronary stents have to be removed complications are frequently encountered placing great strains on patients. The same applies to non-vascular stents as they are placed, for example, into the trachea, esophagus or used in urological applications.

For the above described reasons and from a patient's viewpoint the best stent is still a stent that need not remain inside the body system. Therefore, it is desirable to have available a stent that having fulfilled its purpose can be removed without problems and, in particular, without causing strain on patients.

Such a stent should have the customary stent properties and functions, i.e. be suited to support, expand and stabilize a body lumen. After a given function has been fulfilled the stent should be capable of being removed with minimum expenditure and effort and without leaving any trace of it.

This objective is reached by providing a temporary stent of the kind first mentioned above in which all ring elements start out from a spine extending over the length of the stent and comprise at least two weakened areas, with said areas enabling the ring elements to be collapsible during the process of explantation and said spine being provided with a recovery element located at its proximal end.

The temporary stent according to the invention is designed to be implanted into the body lumen in a customary manner. However, its special design and segments enable this stent to be explanted without difficulty after it has fulfilled its function and via the route by means of which it has entered the body.

The inventive temporary stent is, in particular, a vascular stent suited for implantation into the coronary or peripheral vessel system. However, when appropriately sized, it may as well serve as stent for other body lumens, i.e. in urology for the urinary tracts, the air passages in the area of trachea and bronchial tubes, for the intestinal area, especially the duodenum, for esophagus and bile ducts. Irrespective of these additional capabilities the inventive temporary stent is described hereinafter with reference to a stent employed in the vascular area.

Vascular stents are usually cut from a tubular base material and for implantation purposes reduced to a diameter that enables placement of the stent by means of a guide catheter. For this purpose the stents may be crimped over a balloon that causes them to expand hydraulically at the placement site until the desired diameter has been reached. Alternatively, stents made of shape-memory alloys are used that under the influence of external constraint exerted by a catheter assume a volume-reduced form but expand to the targeted diameter after they are released from the catheter and upon omission of such external constraint. Aside from such a stress-induced transformation to the martensitic phase also a temperature-induced martensitic transformation may be applied or combinations of both. The technical details are known to those skilled in the art.

For the purposes of the invention, the term “proximal” with reference to the stent means facing towards the catheter or attending physician whereas “distal” means upstream of the implantation site facing away from the catheter end.

The term explantation of a stent denotes the recovery or retrieval of a completely implanted stent no longer required as well as the retrieval of a stent that was wrongly placed or became damaged. However, these are primarily stents that having fulfilled their function are no longer needed or have to be replaced otherwise.

Of great significance for the inventive temporary stents are the arrangement of weakened areas in the ring elements and also the recovery element located at the proximal end.

Recovery element and weakened areas are appropriately matched to each other such that when the stent is retracted the ring elements fold in or collapse at their weak points and in this manner enable the stent to be withdrawn into a catheter. The catheter itself may either be a customary guide catheter or a specially designed recovery or retrieval catheter provided at its distal end with a zone of higher strength or stiffness which facilitates the withdrawal and collapsing of the stent.

The inventive temporary stent may be applied in the usual manner, i.e. crimped over a balloon moved to the placement site where the stent is positioned and expanded hydraulically by means of the balloon. Alternatively, shape-memory alloys may also be employed, for example nitinol. Making use of the phenomenon of stress-induced martensitic transformation the stent under the influence of an external constraint exerted by a catheter is transported to the placement site where it is released and, having been liberated, assumes the shape previously impressed on it.

The temporary stents according to the invention are preferably designed such that they are present in the target vessel in fully expanded state. In fully expanded state the ring elements form a more or less regular ring or circle which warrants high stability even in the event the material strength and string width of the stent are low or small. As soon as the ring elements have assumed their full circular shape the weakened areas no longer have any effect when radial loads are applied. Moreover, circular ring elements enable stents to be placed in a simple and safe manner and aside from having adequate radial strength impart sufficient flexibility with respect to adapting to the relevant configuration of the vessel to be treated.

Apart from the necessity that vessels must suffer minimum injury during the implantation process the safe explantation of the stent is also of greatest significance. For that reason, the stent must not be allowed to grow in significantly and, furthermore, must be collapsible in a defined manner. To prevent such an ingrowth it may thus be expedient to coat the stent, in particular also with substances that inhibit the growth of cells. Stents and coatings of this kind are known per se to persons skilled in the art. For example, reference is made in this context to plastic coatings containing a proliferation-inhibiting active agent, for instance rapamycin or Taxol.

During placement the stent is evenly pressed into the wall of the vessel. Moreover, pressing the stent into the vessel wall also serves to secure the stent at the placement site and prevents it from being washed away later. The depth of pressing the stent into the wall amounts half the material thickness. Especially stents impressed in this way may cause the vessel wall to become mechanically injured during stent removal.

To counteract such injuries the outer wall of the inventive stent may be provided with a coating the material of which dissolves under the conditions prevailing at the placement site. Such materials are known and may consist either of plastic or of metal. If the outer coating of the stent has become detached to a significant extent or completely it will be much easier to remove the stent and the danger of the vessel wall suffering injuries reduces significantly.

Expediently, the outer coating of the stent amounts to between 25 and 75% of the stent's entire wall thickness, especially between about 40 and 60%. The wall thickness of vascular stents usually lies in the range of between 40 and 70 μm, if provided with a coating between 50 and 100 μm.

Other than the so-called drug-eluting stents the stents to which the present invention refers are, in view of the fact that they are intended to be explanted, coated only in the outer area with a resorbable material to rule out vessel wall injury to the extent possible. A coating of the inner wall may also be provided consisting of a customary active-agent containing material. In this case the coating of the inner wall and outer wall differs fundamentally with respect to function and purpose.

For example, the coating may be a biologically degradable polymer material as customarily used for stent coatings meant to liberate active agents, Further resorbable materials may also consist of metals capable of dissolving in the circulation, for example magnesium and pure iron. Stents consisting of these materials and dissolving by itself have, for instance, been described in WO 99/003515 A2.

Such a coating of the stent outer wall is as a rule carried out after finishing—electropolishing and cleaning—and pre-crimping. After pre-crimping the elements are located very close to each other. The stent supported on a carrier can then be spray-coated from the outside so that an ideal coating distribution over the outer surface is achieved with a uniform transition towards the elements' side faces. This is especially true for coatings consisting of a plastic material.

In case a retrieval wire has been connected to the stent this wire should also have a coating, at least in its distal area, with said coating to be provided either on one side or all around.

Coatings consisting of a quickly dissolving metal such as pure iron or magnesium may be applied through various methods. For example, tubes consisting of the different materials may be connected with each other before the stent is cut (sandwich technique). Alternatively, the coating may be applied by sputtering or rolled on; a plasma coating method or ultrasonic coating process may be employed especially for magnesium. In case the outer coating consists of a quickly corrodible metal, a sandwich tube or similar element is preferably used for the manufacture of the stent.

The design of the inventive stent providing for a spine or longitudinal element and ring elements projecting from said spine element primarily at right angles offers high flexibility apart from adequate radial strength. The spine is extraordinarily flexible and as such can adapt to the configuration of a vessel in the best possible way. The ring elements emanating from said spine align transversely to the configuration of the vessel which is important for the widening and supporting functions. The expansion of the stent with the ring elements for the main part fully expanded circularly results in the vessel wall being excellently supported and the vessel lumen widened in a defined manner.

The inventive temporary stents have at least two weakened areas, preferably located close to each other at both sides of the spine. In particular, the stent has at least three weakened areas, two close to each other on both sides of the spine and the third located at the side of the ring element opposite the spine. Besides, more than three weakened areas may also be provided of which in any case two are arranged close to the spine and the others distributed in a mirror symmetrical fashion over the circle arc of the respective ring element. However, it has proven particularly useful to provide a stent having three weakened areas located adjacent to and on both sides of the spine with a third one arranged 180° offset to the spine in each ring element.

The stents according to the invention usually extend over a length of 5 to 25 ring elements to suit the respective application. Depending on string width and necessary spacing the number of elements may be greater or smaller. In the coronary field the element spacing generally ranges between 0.5 and 3 mm.

The material thickness in the weakened areas may be lower. Alternatively, the string width of the ring elements in the weakened areas may be reduced. In both cases, the weakening effect is associated with a material reduction which may amount to 50%, preferably be in the range of 20 to 40%.

Moreover, the weakened areas may also be brought about entirely or partly by means of a locally applied heat treatment of these areas. This may, for example, be achieved by treating the relevant areas locally by means of a laser in such a manner that the stent material in the weakened areas is briefly heated to an extent that the strength properties diminish. Heat treatment methods, especially for stainless steel as well as medical stainless steel, are known per se.

With respect to the weakened areas care must be taken that if the stent has been provided with an outer coating consisting of a resorbable material this will neither impair the functional purpose of these weakened areas nor rendered it ineffective. It must be ensured that the weakened areas fulfill their function even after the stent has been coated with the resorbable material.

The stent recovery system provided in accordance with the invention is proximally arranged on the spine. Several embodiments are conceivable with respect to the recovery system. It may thus be designed as an element to which a retriever is attached, for example as a lug, hook or loop. A retriever may be hooked on to this element and in this way enable the stent to be retracted into a recovery catheter. However, the recovery element may also be a retrieval wire directly attached to the spine or hooked on to a lug with said wire remaining on the stent. This variant offers advantages, in particular with vascular stents of small diameter and also with applications where a stent need be placed only for a short period of time. Such a retrieval wire may be very thin, for example having a maximum diameter of 0.3 mm and remains inside the vessel until the stent is recovered. After implantation the guide catheter and the “big” sluices can be removed as a rule. For the temporary guidance of the retrieval wire an indwelling i.v. cannula will be sufficient. Depending on the size of the stent the retrieval catheter may also be of very delicate design having an outer diameter of less than 1 mm, with said catheter usually being introduced through a customary sluice.

However, in the case of a temporary stent for larger-sized vessels and extravascular lumina that variant is given preference which provides for the stent to have only a recovery element to which a retriever can be attached.

It goes without saying that the inventive stents may be provided with radiopaque markers which may be needed. A preferred location for the arrangement of a marker is in the vicinity of the recovery element, or, otherwise, the recovery element may be designed such that it has marker properties.

The stents according to the inventions are manufactured of customary materials, in particular iron (pure iron), stainless steel (medical stainless steel), gold, platinum, shape-memory alloys (nitinol) and tantalum. Stents made of suitable plastic materials can also be employed.

The stents are cut in customary manner from a tubular base material for which purpose laser techniques are especially applied. As a rule, the base material has a wall thickness ranging between 70 and 300 μm, coronary stents after electropolishing between 40 and 70 μm. Due to the fact that the stents usually are expanded to a full circle, high radial strength is achieved exclusively through the shape so that very small wall thicknesses of 50 μm and less are required.

The string width of both the ring elements and the spine is in the range of between 40 and 500 μm, in the case of coronary stents between 40 and 80 μm. Due to the high radial strength of a ring element fully expanded into a circle the string width may be lesser than is usually provided for vascular stents.

In the event that the inventive stents are employed for non-vascular purposes, the material thicknesses and string widths coincide in terms of size and strength with those customarily used within the respective fields of application.

The invention is explained in more detail by way of the enclosed figures where

FIG. 1 shows an inventive temporary stent with ring elements in fully expanded state;

FIG. 2 shows an inventive stent, in the form of a developed view, in manufacturing condition,

FIG. 3 is a detail view of FIG. 2 to provide elucidation on how the ring elements are attached to the spine, and

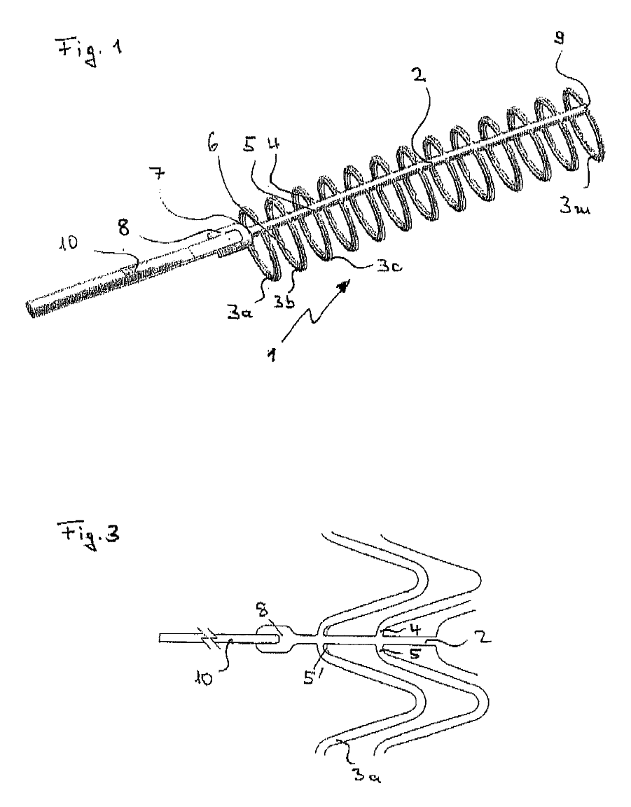

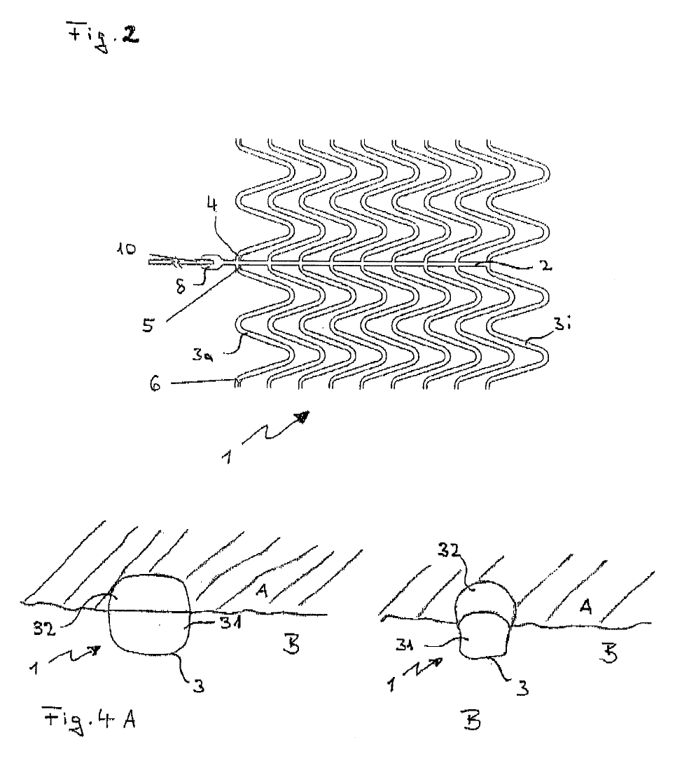

FIG. 4 is a cross-sectional view of a stent design provided with outside coating also providing information on its function.

FIG. 1 shows an inventive temporary stent 1 with its spine 2 and the individual ring elements 3a, 3b, 3c, . . . , 3m in fully expanded state with the ring elements having been expanded into the shape of a full circle. The ring elements 3a . . . 3m are arranged at regular intervals over the length of the stent and connected with each other via the common spine 2. Normally, the ring elements 3a . . . 3m are positioned perpendicularly to spine 2. The string width of ring elements 3 and spine 2 amounts to approx. 50 μm, the material thickness is also in the range of 50 μm.

Each of the ring elements 3 has three weakened areas of which two (4, 5) are arranged immediately adjacent to the spine. In relation to spine 2 a third weakened area 6 is arranged offset by 180°, and within the ring element is thus located opposite spine 2. These weakened areas exist in each of the ring elements 3. The weakened areas 4, 5, 6 are to be viewed as zones where the string width is reduced.

Spine 2 with proximal end 7 and distal end 9 has been provided with a recovery element 8 located at its proximal end 7 with a recovery or retrieval wire 10 being attached to said recovery element. Shown in the figure is that recovery wire 10 is permanently attached to the recovery element 8. In other embodiments the recovery element 8 may be designed as a lug with which a separate retriever engages. For example, the retrieval wire 10 may also be secured directly at the spine 2 by means of a weld spot.

FIG. 2 shows in the form of a developed representation an inventive temporary stent in the production state. During this phase the ring elements 3a . . . 3i have a wavelike configuration and are provided with weakened areas 4, 5, 6, as shown in FIG. 1. At both sides of and immediately adjacent to the spine 2 there are areas of reduced string width 4, 5 where the string width has been reduced by about 40%. Offset by 180° in relation to spine 2 there is a third weakened area 6 the string width of which has also been reduced by 40%.

At the proximal end of spine 2 the recovery element 8 has been shown which connects the retrieval wire 10 to the spine 2.

The stent illustrated in FIG. 2 is cut out of the tube blank in the form shown and for placement purposes crimped onto a customary implantation balloon. In crimped-on condition the wavelike ring elements 3a . . . 3i are fit into each other so that the “wavelength” is reduced and the “amplitude” increased. After implantation the wavelike configuration of the ring elements 3a, . . . , 3i will then be stretched out, i.e. the ring elements 3 have a primarily circular shape, as is shown in FIG. 1.

In FIG. 3 a sectional view of FIG. 2 has been illustrated with spine 2, the ring element 3a and the weakened areas 4, 5 located at both sides of spine 2. 5′ denotes the area of the ring element 3a that was cut off for the purpose of producing the weakened areas.

It is understood that said weakened areas can be produced not only by reducing the material but as well by diminishing the strength of the material. The significant effect is that in the event of a proximally exerted pull force, possibly aided through the recovery catheter mouthpiece, the ring elements 3 are caused to fold in at the weakened spots and in this way permit the entire stent to be retracted into the recovery catheter. The recovery catheter may therefore have a diameter which is significantly lower than that of the implanted stent.

In FIG. 4 the design and functioning of an inventive stent is illustrated which has been provided with an outer coating. A ring element 3 of a stent 1 is shown as a cross-sectional view, with said element 3 consisting on its inside 31 of a durable material, for example stainless steel, nitinol or cobalt/chromium alloy, and being provided on its outside 32 with a coating of a resorbable or corrodible material, for example pure iron or magnesium. The illustration shows ring element 3 of stent 1 in its expanded form and placed at the implantation site with approx. 50% of its thickness being pressed into a vessel wall A while the inner side 31 of the ring element 3 projects into the blood stream B.

FIG. 4b is a cross-sectional view of a ring element 3 showing a stent 1 provided with a polymer coating. The stent body 31 itself—which constitutes the inner side—consists of a customary stent material, for example stainless steel. On its outside the stent has been provided with a coating 32 of a resorbable polymer which covers the outside and becomes thinner towards the flanks. This variant as well projects into the vessel wall A by approx. half of its thickness, whereas the inside 31 of ring element 3 extends into the blood stream B.

The functional purpose provides for the material, which may be a corrodible metal or a resorbable polymer coating, to decompose on its outside 32 under the influence of the blood and immune system more or less quickly so that the retention within vessel wall A diminishes and the element detaches after days or weeks. When the coating material on the outside 32 has been resorbed the stent ran be explanted from the vessel without the risk of causing damage to the wall of the vessel.

1-19. (canceled)

20. A temporary stent comprising:
a spine having a distal end and a proximal end; and
a plurality of ring elements arranged between the proximal and distal ends of the spine, wherein each ring element comprises at least two weakened areas, wherein each ring element is capable of an expanded state and a collapsed state, and wherein the weakened areas are configured to enable each ring element to collapse to its collapsed state.

21. The stent according to claim 20, wherein the plurality of ring elements are arranged parallel to each other.

22. The stent according to claim 20, wherein each ring element is circular when in its expanded state.

23. The stent according to claim 20, wherein each ring element comprises at least three weakened areas.

24. The stent according to claim 23 wherein at least two weakened areas are arranged adjacent to the spine and at least one weakened area is arranged radially opposite the spine.

25. The stent according to claim 20, wherein the weakened areas comprise a lower strength material than a remaining portion of the ring element.

26. The stent according to claim 20, wherein the weakened areas have a lower material thickness than a remaining portion of the ring element.

27. The stent according to claim 26, wherein the lower material thickness is produced at least in part through a local heat treatment.

28. The stent according to claim 20, further comprising a recovery element located at the proximal end of the spine.

29. The stent according to claim 28, further comprising a retriever configured to be coupled to the recovery element.

30. The stent according to claim 28, wherein the recovery element comprises a lug, a hook, or a loop.

31. The stent according to claim 29, wherein the retriever is a retrieval wire.

32. The stent according to claim 20, wherein the stent comprises iron, steel, noble metal, or nitinol material.

33. The stent according to claim 20, wherein the stent is configured to be implanted within a coronary vessel.

34. The stent according to claim 20, wherein the stent has a wall thickness ranging between 20 and 100 μm.

35. The stent according to claim 34, wherein the stent has a wall thickness ranging between 40 and 60 μm.

36. The stent according to claim 20, wherein the stent has a string width ranging between 50 and 500 μm.

37. The stent according to claim 20, further comprising a resorbable outer coating coupled to at least an outer portion of each of the plurality of ring elements.

38. The stent according to claim 37, wherein the resorbable outer coating is a dissolvable material comprising plastic, iron, or magnesium.

39. The stent according to claim 37, wherein each ring element has a wall thickness, and wherein the outer coating is 25 to 75% of the wall thickness.

40. A method for supporting a body lumen comprising:
delivering a temporary stent within the body lumen, the temporary stent comprising a spine having a distal end and a proximal end and a plurality of ring elements arranged between the proximal and distal ends of the spine, wherein each ring element comprises at least two weakened areas configured to enable each ring element to collapse for explantation from the body lumen; and
expanding the ring elements to support the body lumen.

41. The method according to claim 40, further comprising:
collapsing the plurality of ring elements at the at least two weakened areas; and
removing the temporary stent from the body lumen.
